(19) United States
(12) Patent Application Publication
(10) Pub. No.: US 2025/0276045 A1
(43) Pub. Date: Sep. 4, 2025

(54) Composition Containing Collagen

(71) Applicant: YUJIAN International Biotechnology, Inc., Hsinchu (TW)

(72) Inventors: YAO-JEN TU, Hsinchu (TW); YU-SHAN LIU, Hsinchu (TW); YU-TING KU, Hsinchu (TW); CHENG-HAO CHEN, Hsinchu (TW); FONG-JE LIN, Hsinchu (TW); YI-LING YANG, Hsinchu (TW)

(21) Appl. No.: 18/830,046

(22) Filed: Sep. 10, 2024

(30) Foreign Application Priority Data
Mar. 1, 2024 (TW) 113107505

(51) Int. Cl. A61K 38/39; A61K 31/375; A61K 31/728; A61K 35/57; A61K 35/60; A61K 36/45; A61K 36/736; A61K 47/34; A61K 47/36; A61P 17/02

(52) U.S. Cl. CPC A61K 38/39; A61K 31/375; A61K 31/728; A61K 35/57; A61K 35/60; A61K 36/45; A61K 36/736; A61K 47/34; A61K 47/36; A61P 17/02

(57) ABSTRACT

A composition containing collagen, which consists of collagen, hyaluronic acid, vitamin C, indigestible maltodextrin, Emblica officinalis, lingonberry extract, cherry blossom extract, oligomerized polyphenols and flavoring ingredients, wherein per 1000 g, the content of collagen ranges from 500-935 g, the content of hyaluronic acid ranges from 1-5 g, the content of vitamin C ranges from 10-30 g, the content of indigestible maltodextrin ranges from 50-200 g, the content of Emblica officinalis ranges from 2-30 g, the content of lingonberry extract ranges from 1-20 g, the content of cherry blossom extract ranges from 1-20 g, the content of oligomerized polyphenols ranges from 0-20 g, and the content of flavoring ingredients is 0-175 g.

### CROSS-REFERENCE TO RELATED APPLICATIONS

This application claims the benefit of TW Patent Application No. 113107505, filed on Mar. 1, 2024, the content of which are hereby incorporated by reference in their entirety.

### FIELD OF THE INVENTION

The present invention relates to a composition containing collagen with wound repair and skin protection properties, particularly relating to a composition containing collagen suitable for human ingestion.

### BACKGROUND OF THE INVENTION

Collagen is a polymer that constitutes various extracellular matrices. It plays the role of binding tissues in animal cells. It is the most abundant protein in animals, accounting for about 25%-35% of human protein, which is equivalent to it accounts for 6% of the human body weight and is distributed in various tissues and organs throughout the body, such as bones, cartilage, ligaments, skin, cornea, various endometrium, and fascia. Especially in the skin and connective tissues of the human body, it contains a large amount of collagen.

Collagen has the basic characteristics of non-antigenicity, biodegradability, biocompatibility, synergy of bioactive substances, and natural polymer materials, making it widely used in medicine. In addition, collagen has become the most common main ingredient in cosmetics and skin care products. When existing collagen materials are applied to the skin, they can improve the appearance of the skin and protect the structure and function of the skin.

Human skin is mainly composed of epidermis, dermis and hypodermis. The epidermis is composed of the outermost layer of skin tissue. It is a protective layer on the surface of the body that can maintain moisture in the body and provide barrier function to block physical or it is a chemical invasion or to prevent pathogens from entering the human body. When the skin is exposed to external stimuli or is under stress in life, it will trigger the formation of free radicals, and the free radicals will activate metalloproteinases (such as collagenase), so the collagen in the extracellular matrix of the dermal layer of the skin is to be decomposed, and skin aging phenomena will be introduced such as wrinkles and thinning.

### SUMMARY OF THE INVENTION

According to drawbacks of the prior art, the main objective of the present invention is to provide a composition containing collagen. The composition is synthesized from a variety of natural extracts, so it can be taken orally or internal use by humans or animals. It can slow down wound inflammation, repair and wound healing. It has high biocompatibility for human or animals without causing any side effects.

According to the above objective, the present invention provides a composition containing collagen, the composition consists of collagen, hyaluronic acid, vitamin C, indigestible maltodextrin, Emblica officinalis, flavoring ingredients, in which per 1000 grams, the lingonberry extract, cherry blossom extract, oligomerized polyphenol, and the range of the collagen is 500-935 grams, the range of the hyaluronic acid is 1-5 grams, the range of indigestible maltodextrin is 50-200 grams, the range of Emblica officinalis is 2-30 grams, the range of lingonberry extract is 1-20 grams, the range of cherry blossom extract is 1-20 grams, the range of oligomerized polyphenol is 0-20 grams, and the range of flavoring ingredient is 0-175 grams.

### BRIEF DESCRIPTION OF THE DRAWINGS

FIG. 1 is a diagram showing the wound healing rates of different groups of pigs in accordance with the present invention disclosed herein.

FIG. 2 are observation photographs showing the wound healing of pig No. 232 at different time in accordance with the present invention disclosed herein.

FIG. 3 are observation photographs showing the wound healing of pig No. 233 at different time in accordance with the present invention disclosed herein.

FIG. 4 are observation photographs showing the wound healing of pig No. 234 at different time in accordance with the present invention disclosed herein.

FIG. 5 are observation photographs showing the wound healing of pig No. 235 at different time in accordance with the present invention disclosed herein.

### DETAILED DESCRIPTION OF THE PREFERRED EMBODIMENT

In order to make the purpose, technical features and advantages of the present invention better understood by those in the relevant technical field, and able to implement the present invention. The technical contents involved in the embodiments that are well known to those skilled in the art will not be described again.

It should be noted that AGEs are the product of cross-linking reactions caused by reducing sugars in proteins, which will cause the protein to lose their original functions. For example, when the elastin in skin connective tissue produces AGEs, the skin will lose its elasticity; when AGEs are produced in blood vessels, changes in protein structure will cause arteriosclerosis; when AGEs accumulate in the lens and vitreous body of the eye, it will cause eye damage. In addition to the increase in blood sugar, which can cause the accumulation of endogenous AGEs products, the healing process of food and smoking are the main sources of exogenous AGEs. In natural organisms, the Menard reaction in which sugars bind to proteins (such as collagen and elastin in the skin) is common. As age increases, AGEs will accumulate in the skin. When the matrix protein of the skin (such as collagen and elastin) is denatured, they lose their original functions. In this situation, the aging of skin will occur.

The present invention disclosed a composition containing collagen, and other ingredients are combined according to the composition, and the composition containing collagen disclosed in the present invention at least consists of collagen, hyaluronic acid, vitamin C, indigestible maltodextrin, Emblica officinalis, lingonberry extract, cherry blossom extract, oligomerized polyphenols, and flavoring ingredient.

Collagen. Collagen which is fish collagen or hydrolyzed fish collagen. The aspect of using fish collagen or hydrolyzed fish collagen in the present invention is that the particles of the collagen molecules extracted from fish are finer than those extracted from pigs, cows or chicken, and it can be confirmed by the literature. The decomposition rate of fish collagen is 7 times higher than that of pig skin collagen.

It has been pointed out in some research literature that the advantages of either fish collagen or hydrolyzed fish collagen can prevent osteoporosis, deforming arthritis and/or bedsores for bone joints. In addition, it can also promote osteoblast differentiation and bone cell metabolism. In addition, collagen can improve overall muscle mass and increase muscle density. Accordingly, the present invention utilizes fish collagen or hydrolyzed fish collagen as the main collagen source of the composition containing collagen.

Hyaluronic acid. The main source of the hyaluronic acid can be divided into three types: animal extraction, bacterial fermentation and chemical synthesis, in which the animal extraction is mainly extracted from parts such as rooster combs or animal cartilage, but the extraction cost is high. Bacterial fermentation is the fermentation product of epidemic Streptococcus. Safety needs to be paid attention to during the process, and the extraction process needs to be strictly controlled to avoid harming the human body. Chemical synthesis is the artificial synthesis of hyaluronic acid, which is mostly used in medical aesthetic treatments to reduce the risk of allergic reactions and infection after injection. In one embodiment, animal sources or fermented sources are used as the main source, specifically, cockscomb extract is used as the main ingredient. After the cockscomb is obtained, hyaluronic acid can be obtained from the cockscomb using enzyme hydrolysis and salt precipitation. Hyaluronic acid also has the functions of whitening, removing wrinkles, hydrating and preventing hair loss.

Vitamin C. Vitamin C is food-grade vitamin C. The source of vitamin C can be naturally extracted, such as lemon, camu and other plant fruits rich in vitamin C. It can also be obtained through fermentation or synthesis. In the present invention, vitamin C is used to provide the composition containing collagen with whitening and collagen production effect.

Indigestible maltodextrin. The source of indigestible maltodextrin is derived from conversion of corn starch into isomaltodetrin by enzymes, also known as indigestible maltodextrin, which is used to provide composition containing collage with blood sugar stability, reduce glycation effect.

Emblica officinalis. Emblica officinalis is also known as amla or Indian gooseberry. The fresh Emblica officinalis adopts an advanced green and environmentally friendly extraction method. The first step of the extraction method is that the Emblica officinalis is made into juice by cold pressing, and then concentrated and extracted. The manufacturing process does not utilize solvents, excipients or preservatives. Emblica officinalis is a fruit that is used both as medicine and food. When the fresh fruit is first eaten, it tastes astringent and sour. After eating, the aftertaste is sweet and refreshing. In addition, there are a variety of hydrolyzable tannins in Emblica officinalis, mainly amla A and amla B. Some studies have pointed out that the structure of amla A and amla B were analyzed using nuclear magnetic resonance (NMR), which can be redefined as β-glucogallin and Mucic Acid Gallate. To compare with the vitamin C, β-glucogallin is a more effective antioxidant molecule. Therefore, β-glucogallin is added to the composition containing collagen which can provide antioxidant effect through the β-glucogallin in Emblica officinalis.

Lingonberry extract. Lingonberry extract is obtained from cowberry (or lingonberry) (scientific name: Vaccinium vitis-idaea L). Lingonberry is a deciduous shrub plant of the Rhododen Dacease family, it originates from the forest of Eurasia, so it is known as the “Red Bean of the North”. Lingonberry is rich in active nutrients, such as Arbutin, which can effectively reduce the activity of tyrosinase, thereby reducing dark spots and helping to glow with fair skin. Lingonberry is often used in makeup and skin care.

Cherry blossom extract. Cherry blossom is extracted from cherry blossom flowers. The active ingredients in cherry blossom include at least 1-caffeoyl-O-β-D-glucopyranoside, whose chemical formula is as shown in formula (I)

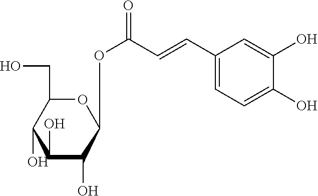

and quercetin glucoside (3-O-β-D-glucopyranoside), whose chemical formula is as shown in formula (II)

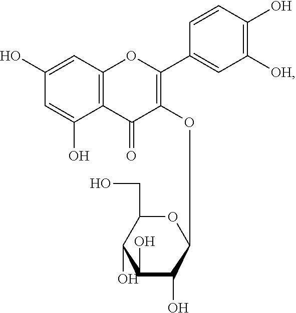

in which caffeinyl glucose accounts for 8%-10% of cherry blossom extract, and quercetin glucoside accounts for 0.4%-0.6% of cherry blossom extract.

Oligomerized polyphenols. Oligomerized polyphenols are stable, soluble small molecule polyphenols formed by combining macromolecular polyphenol polymers in mature lychee fruits (including peel) and combined with green tea catechins after acid decomposition. They can relieve fatigue and soothe muscles. It has effects on menstrual pain and circulation promotion.

Flavoring ingredients. Flavoring ingredients are used to prepare the taste of the composition containing collagen suitable for oral administration. They can be natural flavors, such as natural lemon juice, natural citrus juice powder, natural apple powder, etc., or synthetic flavor cab be used. In another embodiment, the composition containing collagen did not include flavoring ingredients.

According to the above, in one embodiment of the present invention, in per 1000 grams of composition containing collagen, the range of collagen is 500-935 grams, the range of hyaluronic acid is 1-5 grams, the range of indigestible maltodextrin is 50-200 grams, the range of Emblica officinalis is 2-30 grams, the range of lingonberry extract is 1-20 grams, the range of cherry blossom extract is 1-20 grams, the range of oligomerized polyphenol is 0-20 grams, and the range of flavoring ingredient is 0-175 grams.

The present invention also conducted an animal experimentation based on the composition containing collagen prepared above. The animal experimentation is conducted on four pigs, and their numbers are No. 232, No. 233, No. 234, and No. 235 respectively. The purpose of the test is to evaluate the effect of daily addition of composition containing collagen to the feed on animal cortex wound (2±0.5 cm×2±0.5 cm). Three wounds were opened on the left and right sides of animal's spine. The numbers on the right side are R1, R2, and R3 and the numbers on the left side are L1, L2, and L3. The wound healing is observed at each time point within 28 days after the wound opening, and the healing rate is calculated to explore the healing efficacy.

The animal experimentation is conducted with two boars and two sows aged 2-3 months (totally 4 pigs). The reason for choosing pigs of animal testing is that pigs are mammals, the physiological function of pigs are close to those of humans, and the skin thickness of pigs is close to that of humans when pigs are 2-3 months old.

### Animal Room Breeding Condition:

Temperature: 18° C.-26° C., the daily average temperature change does not exceed ±2° C. Relative humidity: 30%-70%. Light cycle: 12 hours of daylight, 12 hours of darkness.

### Preparation of Experimentation Substance (Composition Contain Collagen):

The powdered composition containing collagen is dissolved in 200 ml of drinking water and then is mixed with the feed to obtain the mixture. The mixture is placed in the feed trough for pigs to eat. In this experiment, the surface area formula for humans and pigs is as follows:

Human: BSA (cm²)=0.0235×W^{0.51456}×H^{0.42246} (formula III),
in which W indicates weight (Kg) and H indicates height (cm).

Farm Pigs: BSA (cm²)=0.0734BW_{Kg} (formula IV),
in which symbol BW expresses body weight (Kg). For example, the dose for a human with a height of 170 cm and weight of 60 Kg is 7 g. The required dosage of the composition containing collagen is calculated once a week based on the pig body weight and the surface area formula.

### Administration Approaches and Methods:

The four pigs are divided into two groups, group A and group B. In group A, once a day, 200 ml of drinking water is mixed with feed to place in the feed trough for feeding the pigs. In group B, once a day, 200 ml of drinking water containing the composition containing collagen is mixed evenly with the feed and placed in the feed trough for feeding the pigs.

### Experimentation Method and Sampling:

### Animal Fasting:

Animals are fasted for more than 8 hours the day before incision is operated to form a wound on the skin of animals, and the drinking water is not restricted. Surgical anesthesia: intramuscular injection of Atropine at a dose of 0.04 mg/kg, the intramuscular injection of Telazol at a dose of 6 mg/kg and Rompun at a dose of 2.2 mg/kg followed 10-15 minutes later. Analgesic medication: before incision is performed to form a wound on the skin of animal, Ketoprofen (analgesic) at a dose of 3 mg/kg is first intramuscularly injected which is used for analgesic. Incision surgery: the hair on both side (right side of left side) of the surgical area of the back of the pigs is first shaved. Then, iodine solution is applied to the surgical area to be incised and wipe away the residual iodine solution with 75% alcohol. Lidocaine at a dose of 3 mg/kg is injected subcutaneously into the incision site for local anesthesia. The incision operation started about 5 minutes later, the incision operation is performed with full-thickness square to form wounds, there are three wounds on each side (right side and left side respectively) and numbered respectively.

During experimentation process, wound healing is a series of complex processes that includes cell migration, the formation of granulation tissue, angiogenesis and matrix remodeling, all of which rely on the regulation of the extracellular matrix. In addition, when the wound is in the inflammatory stage, it will be accompanied by symptoms such as redness, swelling, heat, and/or pain. At this time, platelets will reach the wound and react with fibrin to form a clot. It needs to be explained is that the clot is capable of protective effect of preventing body fluids and the wound from being exposed to the outside environment. Platelets will release a large amount of growth factor and inflammatory cell such as neutrophils and macrophages penetrate into the wound for phagocytosis and eliminate pathogens. On the first day of the experimentation, tissue fluid accumulated is observed in each wound of group A and group B respectively. On the 3^{rd} day to the 5^{th} day of the experimentation, it was observed with the naked eye of the human being that the tissue fluid secreted by the wound in group A and group B is changed from liquid to gel, so a protection effect is generated to prevent the wounds from being exposed. Although there was some slight redness, swelling and inflammation around the wounds, but the above phenomena is a reaction in the early stages of normal wound healing. According to above results, oral administration of a composition containing collagen during the inflammatory period has no obvious effect in slowing down wound inflammation. However, the wound healing rate data on the 5^{th} day showed that group B is better than group A, and the difference is statistically significant (P<0.05), showing that the wound healing process in group B has entered into the tissue proliferation stage from the inflammatory stage. Accordingly, it can effectively short the time of the wound is in the inflammatory stage, and the discomfort caused by inflammatory reactions in patients in future clinical application may reduce. The average wound healing rates of different group at different time points are listed in Table 1.

TABLE 1

| | average wound healing rates of different group at different time points (%) | | | | | | | | | | |
|---|---|---|---|---|---|---|---|---|---|---|---|
| group | 0 day | 1 day | 3 day | 5 day | 7 day | 10 day | 14 day | 18 day | 21 day | 24 day | 28 day |
| A | 0.00 | 4.56 ± | 8.08 ± | 6.91 ± | 19.26 ± | 43.85 ± | 62.48 ± | 72.61 ± | 76.31 ± | 78.74 ± | 84.96 ± |
| (control | | 2.21 | 2.27 | 1.25 | 3.04 | 5.09 | 2.40 | 1.29 | 2.63 | 2.19 | 1.14 |
| group) | | | | | | | | | | | |
| B | 0.00 | 4.80 ± | 7.15 ± | 14.55 ± | 26.78 ± | 58.34 ± | 75.71 ± | 79.62 ± | 86.53 ± | 91.58 ± | 94.33 ± |
| (test group) | | 1.36 | 2.37 | 2.92* | 3.38 | 2.89* | 2.22** | 1.84** | 2.33* | 2.14** | 1.25** |

In Table 1, n = 12 for each group from 0 day to 18^{th} day, n = 6 for each group on the 21^{st}, 24^{th} and 28^{th} day. Data are presented as mean ± SEM. In Table 1, the symbol “*” means P < 0.05 and the symbol “**” means P < 0.01.

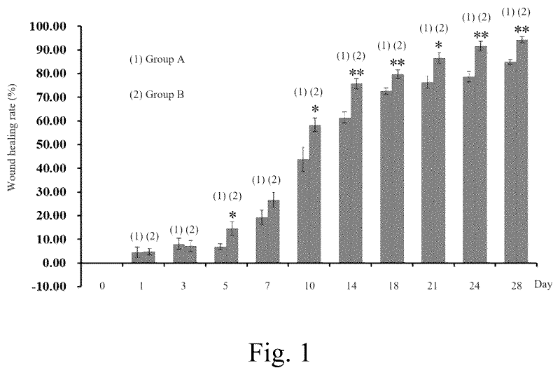

FIG. 1 is a graph of wound healing rate of different groups of pigs. The data are present as mean±SEM and analyzed using independent sample t test. The data marked with symbol “*” in the observation time point which indicates a significant difference between the two groups (P<0.05). The data marked with symbol “**” in the observation time, which indicates that there is a highly significant difference between the two groups (P<0.01).

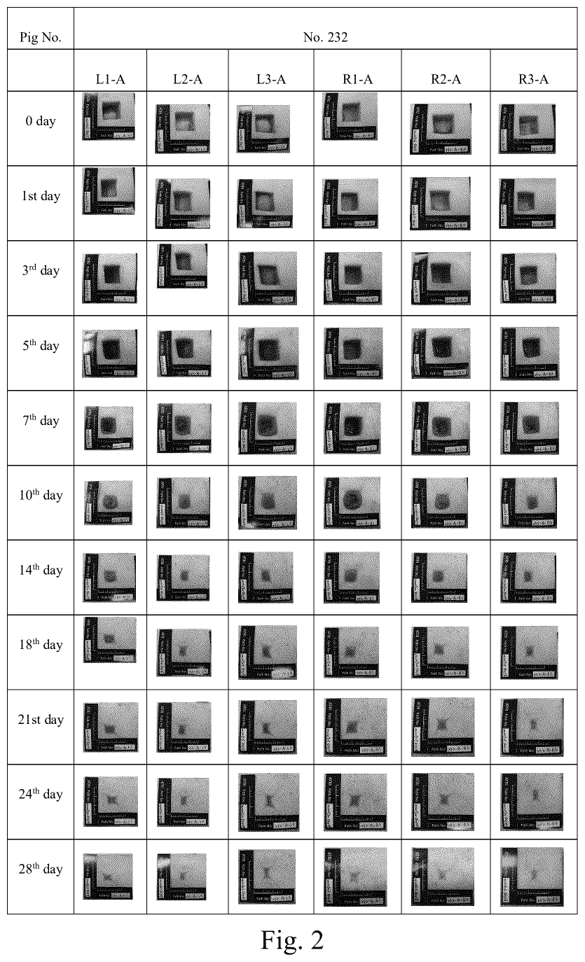

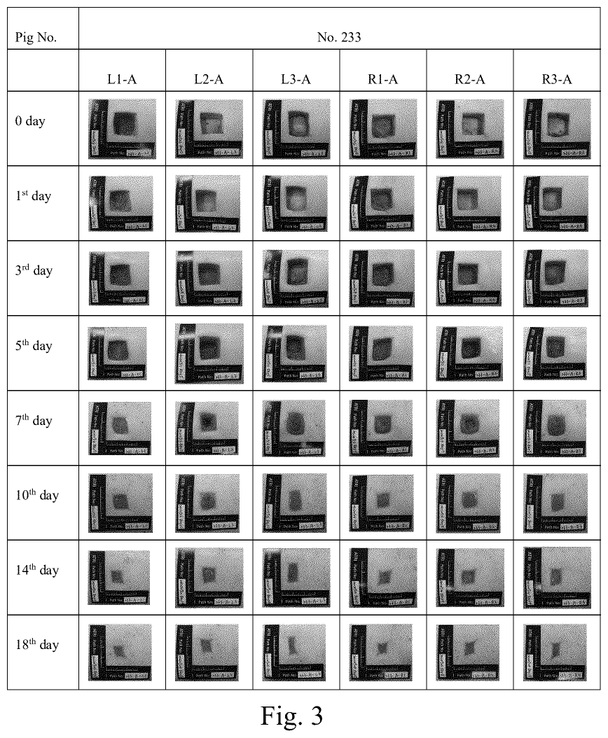

Two of four pigs are organized as No. 232 and No. 233 and grouped into group A, another two of four pigs are organized as No. 234 and No. 235 and grouped into group B, and the wound healing conditions of each group A and group B and each pig is observed at different time, as shown in FIG. 2 to FIG. 5. In FIG. 2 and FIG. 3, FIG. 2 are observation photographs showing the wound healing of pig No. 232 at different time, and FIG. 3 are observation photographs showing the wound healing of pig No. 233 at different time. It can be seen from FIG. 2 and FIG. 3 that the wounds of pig No. 232 and pig No. 233 gradually shrank and healed as time went by. The wound healing rate (%) of pig No. 232 and pig No. 233 at different time are listed in Table 2.

TABLE 2
Group A-wound healing rate (%)

| | | Pig No. | | | | | | | | | | | |
|---|---|---|---|---|---|---|---|---|---|---|---|---|---|
| | | No. 232 | | | | | | No. 233 | | | | | |
| | | Wound site | | | | | | | | | | | |
| time | | L1 | L2 | L3 | R1 | R2 | R3 | L1 | L2 | L3 | R1 | R2 | R3 |
| 0 | day | 0.00 | 0.00 | 0.00 | 0.00 | 0.00 | 0.00 | 0.00 | 0.00 | 0.00 | 0.00 | 0.00 | 0.00 |
| 1 | day | 14.32 | 4.63 | 11.34 | −0.33 | 16.94 | 5.29 | 12.93 | 0.68 | −6.61 | −2.67 | 0.34 | −2.15 |
| 3 | days | 19.51 | 8.51 | 13.52 | 11.33 | 12.66 | 12.29 | 10.11 | 11.07 | 8.10 | −2.16 | 1.46 | −9.48 |
| 5 | days | 12.03 | 4.41 | 4.10 | 2.62 | 2.18 | 13.25 | 8.99 | 3.35 | 4.64 | 14.81 | 6.25 | 6.27 |
| 7 | days | 18.50 | 19.48 | 19.88 | −4.72 | 11.11 | 16.47 | 39.17 | 25.84 | 16.05 | 29.54 | 22.64 | 17.10 |
| 10 | days | 31.30 | 52.55 | 51.67 | −3.81 | 39.92 | 54.73 | 57.85 | 34.93 | 44.97 | 63.14 | 47.15 | 17.10 |
| 14 | days | 51.30 | 71.12 | 68.43 | 43.63 | 60.76 | 68.43 | 69.65 | 60.07 | 57.39 | 68.65 | 59.41 | 58.93 |
| 18 | days | 70.05 | 74.11 | 78.29 | 61.96 | 70.05 | 75.53 | 77.74 | 68.70 | 72.73 | 74.32 | 72.71 | 75.13 |
| 21 | days | 72.94 | 78.33 | 81.03 | 64.65 | 80.15 | 80.73 | Euthanasia sampling | | | | | |
| 24 | days | 73.70 | 78.40 | 80.62 | 71.96 | 81.02 | 86.72 | | | | | | |
| 28 | days | 81.86 | 83.75 | 85.70 | 82.56 | 86.60 | 89.30 | | | | | | |

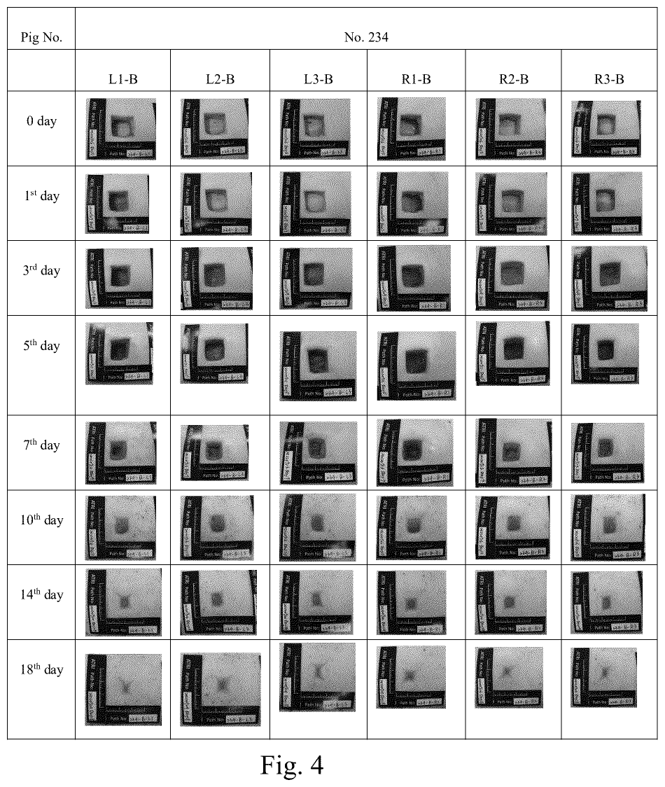

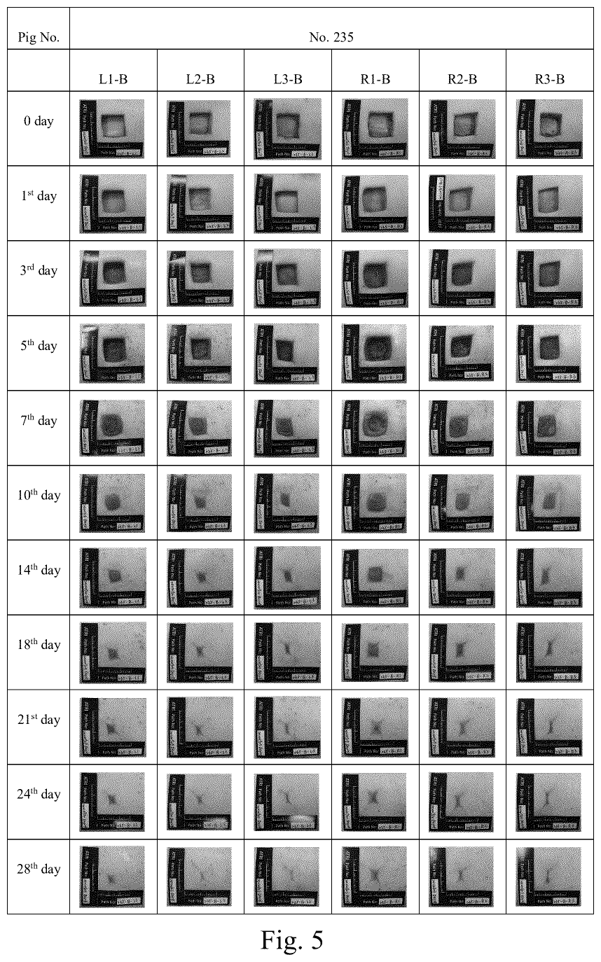

In FIG. 4 and FIG. 5, FIG. 4 are observation photographs showing the wound healing of pig No. 234 at different time, and FIG. 5 are observation photographs showing the wound healing of pig No. 235 at different time. It can be seen from FIG. 4 and FIG. 5 that the wound of pig No. 234 and pig No. 235 gradually shrank and healed as time went by. The wound healing rate (%) of pig No. 234 and pig No. 235 at different time are listed in Table 3.

TABLE 3
Group B-wound healing rate (%)

| | | Pig No. | | | | | | | | | | | |
|---|---|---|---|---|---|---|---|---|---|---|---|---|---|
| | | No. 234 | | | | | | No. 235 | | | | | |
| | | Wound site | | | | | | | | | | | |
| time | | L1 | L2 | L3 | R1 | R2 | R3 | L1 | L2 | L3 | R1 | R2 | R3 |
| 0 | day | 0.00 | 0.00 | 0.00 | 0.00 | 0.00 | 0.00 | 0.00 | 0.00 | 0.00 | 0.00 | 0.00 | 0.00 |
| 1 | day | 12.07 | 8.84 | 10.02 | 7.63 | −0.74 | 7.77 | 4.99 | 1.48 | 3.99 | −3.74 | 0.84 | 4.46 |
| 3 | days | 10.74 | 11.66 | 4.65 | 5.97 | −5.88 | 3.83 | 13.54 | 21.53 | 16.37 | 1.95 | 6.93 | −5.46 |
| 5 | days | 12.73 | 15.97 | 23.19 | 23.51 | 13.24 | 21.70 | 11.68 | 22.18 | 24.76 | −8.81 | 13.70 | 0.70 |
| 7 | days | 36.02 | 24.57 | 31.94 | 27.92 | 29.87 | 30.77 | 15.16 | 40.27 | 38.32 | −3.11 | 27.79 | 21.88 |
| 10 | days | 56.51 | 43.97 | 59.32 | 57.80 | 56.08 | 57.83 | 60.52 | 72.95 | 74.23 | 38.40 | 61.24 | 61.27 |
| 14 | days | 76.26 | 67.59 | 71.47 | 71.70 | 69.31 | 85.41 | 73.34 | 82.95 | 85.34 | 61.82 | 80.69 | 82.67 |
| 18 | days | 82.53 | 72.75 | 79.90 | 74.68 | 76.41 | 72.55 | 79.69 | 87.55 | 89.14 | 72.66 | 77.90 | 89.71 |
| 21 | days | Euthanasia sampling | | | | | | 82.71 | 90.30 | 93.30 | 78.54 | 83.62 | 90.96 |
| 24 | days | | | | | | | 88.79 | 96.02 | 96.85 | 83.15 | 90.15 | 94.51 |
| 28 | days | | | | | | | 92.00 | 96.35 | 98.04 | 89.83 | 93.65 | 96.14 |

When the wound is in the tissue proliferation stage, the inflammatory cells secrete cytokines and growth factors to activate the migration of fibroblasts and myofibroblasts and begin to synthesize collagen to support the basal layer of new tissue so as to promote the regeneration and repair of the dermis and epidermis. On the 7^{th} day of the experimentation, the wound is in the initial stage of tissue proliferation. It can be observed with the naked eye that the wounds in group A and group B began to shrink and heal, and granulations is produced. However, the granulations generated at this stage had not yet filled the entire wound. According to the judgement of the degree of wound healing, the group B is better than group A. On the 10^{th} day of the experimentation, the wound was in the middle stage of tissue proliferation at this time. It can be obviously to observe the granulation is generated in each wound of group A and group B, and the shrink of wound is significant. The data of wound healing rate of the group B is better than that of group A. The difference is statistically significant (P<0.05), except for the granulation of wound sites L1 and R1 of pig No. 232 in group A, which has not filled the wound and is raised obviously, the remaining wounds in both groups are filled with granulation tissue. Under naked eye observation, the wounds in group B are flatter than those in group A, which indicates a better degree of re-epithelialization. On the 14^{th} day of the experimentation, all wounds in groups A and B had been filled with granulation tissue and are in a flat state and the wounds shrunk rapidly, so the wound healing rate data showed that group B is better than group A, and the difference is statistically significant (P<0.01). On the 18^{th} day of the experimentation, the wound is in the late stage of tissue proliferation. Under naked eye observation, the wound in group B continued to shrink rapidly and the color of wound is lighter, which may mean that it has entered the process of collagen stacking, and the wound healing rate in group B was high. In group A, the difference is statistically significant (P<0.01). On the 18^{th} day of the experimentation, one pig from each group (Group A: No. 233, Group B: No. 234) is sacrificed for histopathological analysis. The 21^{st} to 28^{th} day of the experimentation is the wound healing and remodeling period. During this stage, the wound will continue to produce a large amount of collagen and continue to increase the fiber strength until it is fully stable. In terms of the wound healing rate evaluation, group B is better than the group A from the 21^{st} day to 28^{th} day of the experimentation, and the difference is statistically significant (P<0.05).

Table 4 shows the pathological data of No. 233 and No. 234 on the 18^{th} day.

TABLE 4

| Pig No. | | | | | | | | | | | | |
|---|---|---|---|---|---|---|---|---|---|---|---|---|
| — | No. 233 | | | | | | No. 234 | | | | | |
| Group | | | | | | | | | | | | |
| — | Control | | | | | | Test | | | | | |
| Necropsy day | | | | | | | | | | | | |
| 19 | 19 | | | | | | 19 | | | | | |
| Animal fate | | | | | | | | | | | | |
| T | T | | | | | | T | | | | | |
| Treatment | | | | | | | | | | | | |
| None | Dermal excision, full thickness | | | | | | | | | | | |
| Section ID | | | | | | | | | | | | |
| Blank | L1 | L2 | L3 | R1 | R2 | R3 | L1 | L2 | L3 | R1 | R2 | R3 |

H&E stain, Semi-quantitative evaluation of tissue regeneration.

| | | | | | | | | | | | | | |
|---|---|---|---|---|---|---|---|---|---|---|---|---|---|
| Epithelialization^{a} | 4 | 3 | 3 | 2 | 3 | 1 | 1 | 4 | 3 | 4 | 3 | 4 | 4 |
| Neovascularization^{a} | — | 3 | 2 | 3 | 2 | 1 | 2 | 2 | 3 | 1 | 3 | 3 | 3 |
| Fibroblasts^{a} | — | 4 | 4 | 4 | 4 | 4 | 4 | 4 | 4 | 4 | 4 | 4 | 4 |

MT stain, semi-quantitative evaluation of tissue regeneration

| | | | | | | | | | | | | | |
|---|---|---|---|---|---|---|---|---|---|---|---|---|---|
| Collagen^{a} | — | 2 | 1 | 1 | 2 | 2 | 2 | 3 | 1 | 2 | 2 | 3 | 2 |

H&E stain, Semi-quantitative evaluation of inflammatory response.

| | | | | | | | | | | | | | |
|---|---|---|---|---|---|---|---|---|---|---|---|---|---|
| PMNL^{b} | 1 | 2 | 2 | 3 | 2 | 3 | 1 | 1 | 3 | 0 | 2 | 0 | 3 |
| Lymphocytes^{b} | 1 | 2 | 2 | 3 | 3 | 3 | 3 | 2 | 3 | 3 | 3 | 2 | 2 |
| Plasma cells^{b} | 0 | 0 | 0 | 0 | 0 | 0 | 0 | 0 | 0 | 0 | 0 | 0 | 0 |
| Macrophages^{b} | 1 | 1 | 2 | 2 | 3 | 3 | 3 | 3 | 3 | 3 | 2 | 3 | 2 |
| Giant cell^{b} | 0 | 2 | 1 | 3 | 2 | 2 | 1 | 1 | 1 | 1 | 1 | 1 | 1 |
| Necrosis^{b} | 0 | 2 | 2 | 2 | 2 | 2 | 2 | 0 | 1 | 0 | 0 | 0 | 0 |

H&E stain, semi-quantitative recording of other histopathological observation.

| | | | | | | | | | | | | | |
|---|---|---|---|---|---|---|---|---|---|---|---|---|---|
| Dermal granulation | 0 | 3 | 3 | 3 | 3 | 3 | 3 | 3 | 3 | 3 | 3 | 3 | 3 |
| tissue^{c} | | | | | | | | | | | | | |
| Hemorrhage. foci^{c} | 0 | 2 | 2 | 2 | 2 | 0 | 2 | 2 | 0 | 1 | 0 | 1 | 0 |
| Foreign body fragments, | 0 | 0 | 0 | 0 | 0 | 0 | 0 | 0 | 0 | 0 | 0 | 0 | 0 |
| hair^{c} | | | | | | | | | | | | | |
| Abscess, hypodermis^{c} | 0 | 0 | 0 | 0 | 0 | 0 | 0 | 0 | 0 | 0 | 0 | 0 | 0 |

Dermal wound healing categories.

| | | | | | | | | | | | | | |
|---|---|---|---|---|---|---|---|---|---|---|---|---|---|
| Wound healing stage^{d} | 0 | 2 | 2 | 2 | 2 | 2 | 2 | 3 | 2 | 3 | 2 | 3 | 2 |

In Table 4, the abbreviation T represents terminal sacrifice, the superscription a represents the evaluation standard of semi-quantitative evaluation of tissue regeneration, the superscription b represents the evaluation standard of semi-quantitative evaluation of inflammatory response, the superscription c represents the evaluation standard of semi-quantitative recording of other histopathological observation, and the superscription d represents the evaluation standard of Dermal wound categories respectively. The number 0, 1, 2, 3, 4 represent the scale or score in Table 4 which are arranged according to the subsequent Tables 5-8.

For the evaluation standard of semi-quantitative evaluation of tissue regeneration, the primary area of interest (AOI) focused on epithelium regeneration according to the reference (Gal et al., 2008), the modified 5-phase scoring system used in this study listed as shown in Table 5.

TABLE 5

| | | | Fibroblast/ |
|---|---|---|---|
| Scale | Epithelization | Neovascularization | Collagen |
| 0 | Thickness of cut edges/plf | Absent/phf | Absent |
| 1 | Migration of cells (<50%)/plf | 1 to 3 buds/phf | Minimal/GT |
| 2 | Migration of cells (>50%)/plf | 4 to 7 buds/phf | Mild/GT |
| 3 | Bridging the excision/plf | Broad band/phf | Marked-GT |
| 4 | Keratinization/plf | Extensive band/phf | Marked-GT |

In Table 5, the ST represents surrounding tissue, GT represents granulation tissue, phf represents per high-powered field (400×), and plf represents per low-powered field) (40×).

For the evaluation standard of semi-quantitative evaluation of inflammatory response, the secondary AOI aimed at inflammatory cell response and cell types based on the histopathology evaluation format in accordance with Table E1 from ISO 10993-6:2016, which listed in Table 6.

TABLE 6

| Score | PMNL | Lymphocytes | Plasma cells | Macrophages | Giant cells | Necrosis |
|---|---|---|---|---|---|---|
| 0 | 0/phf | 0/phf | 0/phf | 0/phf | 0/phf | 0/phf |
| 1 | 1 to 5/phf | 1 to 5/phf | 1 to 5/phf | 1 to 5/phf | 1 to 2/phf | Minimal |
| 2 | 5 to 10/phf | 5 to 10/phf | 5 to 10/phf | 5 to 10/phf | 3 to 5/phf | Mild |
| 3 | — | — | Heavy infiltrate | — | — | Moderate |
| 4 | Packed | Packed | Packed | Packed | Sheets | Severe |

In Table 6, PMNL represents polymorphonuclear cells, and phf represents per high-powered field (400×).

For the evaluation standard of semi-quantitative recording of other histopathological observation, in Table 7, the third AOI targeted at other lesions within the defect according to the reference (Mann et al., 2012), the 5-phase scoring system used was.

TABLE 7

| Scale | Description | Definition |
|---|---|---|
| 0 | Within | Tissue considered to be normal, under the conditions |
| | normal | of the study and considering the age, sex, and strain |
| | limits | of the animal concerned |
| 1 | Minimal | The amount of changes barely exceeds that which is |
| | | considered to be within normal limits |
| 2 | Mild/slight | In general, the lesions is easily identified but of |
| | | limited severity |
| 3 | Moderate | The lesion is prominent, but there is significant |
| | | potential for increased severity |
| 4 | Severe | The degree of change is as complete possible |
| | | (occupies the majority of the organ) |

For the evaluation standard of dermal wound categories, based on the reference (Diegelmann et al., 2004), the wound healing status was categorized via 5-phase scoring system as shown in Table 8.

TABLE 8

| Phase | Description | Definition |
|---|---|---|
| 0 | Normal | Without any trauma |
| 1 | Hemostasis | As the blood components spill into the site of |
| | | injury, the platelets come into with exposed |
| | | collagen and other elements of the extracellular |
| | | matrix |
| 2 | Inflammation | The neutrophils enter the wound site and begin |
| | | the critical task of phagocytosis to remove foreign |
| | | materials, bacteria and damaged tissue. As part of |
| | | this inflammatory phase, the macrophages appear |
| | | and continue the process of phagocytosis. |
| 3 | Proliferation | Once the wound site is cleaned out, fibroblasts |
| | | migrate in to begin the proliferative phase and |
| | | deposit new extracellular matrix. |
| 4 | Remodeling | The new collagen matrix then becomes cross- |
| | | linked and organized during the final remodeling |
| | | phase |

According to the pathological data in Table 4, the comprehensive histopathological records and wound healing status on 18^{th} day are summarized in Table 9.

TABLE 9

| Group | control | Test |
|---|---|---|
| Necropsy on study day | 19 | 19 |
| Oral administration | Normal diet | Composition |
| | | containing |
| | | collagen |

| Tissue | | | | | |
|---|---|---|---|---|---|
| regeneration^{a} | | | | (N/N)¹ | |
| | Epithelization | Keratinization | 0/6 | | 4/6 |
| | neovascularization | ≥4 to 7 buds | 5/6 | | 5/6^{NS} |
| | Fibroblasts | ≥Moderate-GT | 6/6 | | 6/6^{NS} |
| | Collagen | ≥Mild-GT | 4/6 | | 5/6^{NS} |

| Inflammatory | | | | | |
|---|---|---|---|---|---|
| response^{b} | | | | (N/N)¹ | |
| | PMNL | >5 to 10/phf | 5/6 | | 3/6^{NS} |
| | Lymphocytes | >5 to 10/phf | 6/6 | | 6/6^{NS} |
| | plasma cell | >5 to 10/phf | 0/6 | | 0/6^{NS} |
| | Macrophages | >5 to 10/phf | 5/6 | | 6/6^{NS} |
| | Giant cell | >3 to 5/phf | 4/6 | | 0/6^{NS} |
| | Necrosis | >Mild | 6/6 | | 0/6^{NS} |

| Other findings^{c} | | | | (N/N)¹ | |
|---|---|---|---|---|---|
| | Dermal granulation | ≥Moderate | 6/6 | | 6/6^{NS} |
| | tissue | | | | |
| | Hemorrhage, Foci | ≥Mild | 5/6 | | 1/6* |

| Dermal | | | | | |
|---|---|---|---|---|---|
| wound healing | | | | | |
| categories^{d} | | | | (N/N)¹ | |
| | wound healing | ≥Proliferation | 0/6 | | 3/6^{NS} |
| | stage | | | | |

Similarly, in Table 9, the superscription a represents the evaluation standard of semi-quantitative evaluation of tissue regeneration, the superscription b represents the evaluation standard of semi-quantitative evaluation of inflammatory response, the superscription c represents the evaluation standard of semi-quantitative recording of other histopathological observation, and the superscription d represents the evaluation standard of dermal wound categories respectively. The symbol “*/**” represents P<0.05/0.01; and NS represents no significant.

As shown in Table 9, the epithelialization and keratinization of six wounds of the control group (normal diet) (pig No. 233) were 0. The six wounds of the test group (pig No. 234) reached epithelialization and keratinization, that is, remolding, and there are a total of 4 wounds. That is to say, in the final remodeling stage, the wound will be reorganized due to the oral administration of the composition containing collagen of the present invention, and the collagen matrix in the composition will be cross-linked with the wound (or skin) to allow the wound to reach epithelialization and keratinization. Naturally, the phenomenon of epithelialization and keratinization will not occur in the control group that does not take the composition containing collagen of the present invention. This also means that applying the composition containing collagen of the present invention to a wound can accelerate wound healing. In addition, from above Table 4, it can be seen that during the inflammatory reaction, in the experimental group (pig No. 234) given a composition containing collagen, five of the six wounds had no necrosis, which greatly reduced the occurrence of necrosis. Table 10 shows the pathological data of No. 232 and No. 235 on the 28^{th} day.

TABLE 10

| Pig No. | | | | | | | | | | | |
|---|---|---|---|---|---|---|---|---|---|---|---|
| No. 232 | | | | | | No. 235 | | | | | |
| Group | | | | | | | | | | | |
| Control | | | | | | Test | | | | | |
| Corpse Collection Day | | | | | | | | | | | |
| 29 | | | | | | 29 | | | | | |
| Animal Fate | | | | | | | | | | | |
| T | | | | | | T | | | | | |
| Treatment | | | | | | | | | | | |
| Dermal excision, full thickness | | | | | | | | | | | |
| Section ID | | | | | | | | | | | |
| L1 | L2 | L3 | R1 | R2 | R3 | L1 | L2 | L3 | R1 | R2 | R3 |

H&E stain, Semi-quantitative evaluation of tissue regeneration.

| | | | | | | | | | | | | |
|---|---|---|---|---|---|---|---|---|---|---|---|---|
| Epitheliali- | 4 | 4 | 4 | 2 | 3 | 4 | 4 | 4 | 4 | 4 | 4 | 4 |
| zation^{a} | | | | | | | | | | | | |
| Neovasculari- | 4 | 4 | 3 | 4 | 3 | 4 | 3 | 3 | 1 | 3 | 3 | 2 |
| zation^{a} | | | | | | | | | | | | |
| Fibroblasts | 4 | 4 | 4 | 3 | 3 | 4 | 4 | 4 | 4 | 4 | 4 | 4 |

MT stain, Semi-quantitative evaluation of tissue regeneration.

| | | | | | | | | | | | | |
|---|---|---|---|---|---|---|---|---|---|---|---|---|
| Collagen^{a} | 3 | 3 | 3 | 2 | 3 | 2 | 4 | 4 | 4 | 3 | 4 | 4 |

H&E stain, Semi-quantitative evaluation of inflammatory response.

| | | | | | | | | | | | | |
|---|---|---|---|---|---|---|---|---|---|---|---|---|
| PMNL^{b} | 2 | 1 | 0 | 3 | 3 | 0 | 0 | 0 | 0 | 0 | 1 | 2 |
| Lymphocytes^{b} | 3 | 2 | 2 | 2 | 3 | 2 | 1 | 1 | 1 | 1 | 1 | 2 |
| Plasma cells^{b} | 0 | 0 | 0 | 0 | 0 | 0 | 0 | 0 | 0 | 0 | 0 | 0 |
| Macrophages^{b} | 3 | 3 | 1 | 3 | 3 | 2 | 2 | 2 | 2 | 1 | 1 | 3 |
| Giant cells^{b} | 3 | 2 | 0 | 0 | 1 | 3 | 3 | 1 | 0 | 0 | 2 | 3 |
| Necrosis^{b} | 2 | 2 | 1 | 2 | 0 | 0 | 0 | 0 | 0 | 0 | 0 | 0 |

H&E stain, Semi-quantitative recording of other histopathological
observation.

| | | | | | | | | | | | | |
|---|---|---|---|---|---|---|---|---|---|---|---|---|
| Dermal | 3 | 3 | 3 | 3 | 3 | 3 | 3 | 3 | 2 | 3 | 2 | 2 |
| granulation | | | | | | | | | | | | |
| tissue^{c} | | | | | | | | | | | | |
| Bleeding, foci^{c} | 2 | 2 | 2 | 2 | 2 | 2 | 1 | 1 | 1 | 2 | 1 | 2 |
| Foreign body | 0 | 0 | 0 | 0 | 2 | 0 | 0 | 0 | 0 | 0 | 0 | 0 |
| debris, hair^{c} | | | | | | | | | | | | |
| Abscess, | 0 | 2 | 0 | 0 | 2 | 2 | 0 | 2 | 0 | 0 | 0 | 2 |
| hypodermis^{c} | | | | | | | | | | | | |

Dermal wound healing categories.

| | | | | | | | | | | | | |
|---|---|---|---|---|---|---|---|---|---|---|---|---|
| Wound healing | 2 | 3 | 3 | 3 | 2 | 3 | 4 | 4 | 4 | 3 | 4 | 4 |
| stage^{d} | | | | | | | | | | | | |

In Table 10, the superscription a represents the evaluation standard of semi-quantitative evaluation of tissue regeneration, the superscription b represents the evaluation standard of semi-quantitative evaluation of inflammatory response, the superscription c represents the evaluation standard of semi-quantitative recording of other histopathological observation, and the superscription d represents the evaluation standard of dermal wound categories respectively.

Similarly, in Table 10, the superscription a represents the evaluation standard of semi-quantitative evaluation of tissue regeneration, the superscription b represents the evaluation standard of semi-quantitative evaluation of inflammatory response, the superscription c represents the evaluation standard of semi-quantitative recording of other histopathological observation, and the superscription d represents the evaluation standard of dermal wound categories respectively. According to the pathological data in Table 10, on the 28^{th} day, the comprehensive histopathological records and wound healing status are listed in Table 11.

TABLE 11

| Group | Control | Test |
|---|---|---|
| Necropsy on study day | 29 | 29 |
| Oral administration | Normal | Composition |
| | diet | containing |
| | | collagen of the |
| | | present invention |

| Tissue | | | | | |
|---|---|---|---|---|---|
| regeneration^{a} | | | | (N/N) | |
| | Epithelization^{a} | Keratinization | 4/6 | | 6/6^{NS} |
| | Neovascularization^{a} | Extensive band | 4/6 | | 0/6* |
| | Fibroblasts^{a} | ≥Moderate-GT | 6/6 | | 6/6^{NS} |
| | Collagen | Marked-GT | 0/6 | | 5/6** |

| Inflammatory | | | | | |
|---|---|---|---|---|---|
| response^{b} | | | | (N/N)¹ | |
| | PMNL | >5 to 10/phf | 3/6 | | 1/6 |
| | Lymphocytes | >5 to 10/phf | 6/6 | | 1/6* |
| | Plasma cell | >5 to 10/phf | 0/6 | | 0/6^{NS} |
| | Macrophages | >5 to 10/phf | 5/6 | | 4/6^{NS} |
| | Giant cell | >3 to 5/phf | 3/6 | | 3/6^{NS} |
| | Necrosis | >Mild | 3/6 | | 0/6^{NS} |

| Other findings^{c} | | | | (N/N)^{a} | |
|---|---|---|---|---|---|
| | Dermal granulation | ≥Moderate | 6/6 | | 3/6^{NS} |
| | tissue | | | | |
| | Hemorrhage, foci | ≥Mild | 6/6 | | 2/6* |

| Dermal | | | | | |
|---|---|---|---|---|---|
| wound healing | | | | | |
| categories^{d} | | | | (N/N)^{a} | |
| | Wound healing | Remodeling | 0/6 | | 5/6** |
| | stage | | | | |

In Table 11, the superscription a represents the evaluation standard of semi-quantitative evaluation of tissue regeneration, the superscription b represents the evaluation standard of semi-quantitative evaluation of inflammatory response, the superscription c represents the evaluation standard of semi-quantitative recording of other histopathological observation, and the superscription d represents the evaluation standard of dermal wound categories respectively. The symbol “*/**” represents P<0.05/0.01 and NS represents no significant.

As shown in Table 11, among the six wounds of the control group (normal diet) (pig No. 232), four wounds reached epithelialization and keratinization, while the six wounds of the experimental group (pig No. 235) have reached epithelialization and keratinization. Similarly, in the final remodeling state, the wound will be even due to the oral administration of the composition containing collagen of the present invention, the collagen matrix will be cross-linked with the wound (or skin), so as to allow the wound to reach epithelialization and keratinization. However, in the control group that does not take the composition containing collagen of the present invention orally, the wound will not reach epithelialization and keratinization. This also means that the degree of the wound healing can be accelerated by using the composition containing collagen of the present invention. In addition, from Table 10, it can be seen that during the inflammatory reaction, there is no necrosis in six wounds of the experimental group (pig No. 235) given the composition containing collagen and significantly reduced the occurrence of necrosis.

The results of histopathological analysis show that in the process of wound healing, the granulation tissue bleeding is mainly due to the microvascular rupture caused by unstable environment or poor neovascularization. However, on the 18^{th} day, group B can strengthen the development of micro vessels in the granulation tissue and alleviate granulation tissue bleeding. Observation of inflammation indicators, which showed that group B did not increase immune cell infiltration on the 18^{th} day and 28^{th} day, it showed that the inflammatory response will not be increased in wounds of group B. Evaluating the tissue regeneration on 18^{th} day, the degree of re-epithelialization in group B is higher than that in group A, and the difference is statistically significant (P<0.05), it indicates that the experimental substance could accelerate wound healing. Although there was no significant difference between the two groups on the 28^{th} day, the degree of re-epithelialization in group B was still higher than that in group A, it indicates that the wounds in group B entered the remodeling phase faster. On the other hand, for the evaluation of collagen production, on the 18^{th} day, the content of newly synthesized collagen in the granulation tissue of group A ranged from very little to minor, while group B stimulated more collagen deposition, and the content of newly synthesized collagen increased from very small to moderate, but the number of samples are limited so significant difference is not achieved. On the 28^{th} day, the content of newly synthesized collagen in the granulation tissue of group A ranged from trace to moderate. New dermal particles cross-linked with extracellular matrix have been found in the granulation tissue of group B, indicating that the wound had entered the remodeling stage, which is important evidence that the experimental substance can promote wound healing.

Animal tests and pathological analysis results show that oral administration of a composition containing collagen for 28 consecutive days can effectively promote wound healing.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A composition containing collagen, which consists of collagen, hyaluronic acid, vitamin C, indigestible maltodextrin, Emblica officinalis, lingonberry extract, cherry blossom extract, oligomerized polyphenol, and flavoring ingredients, wherein per 1000 grams, the content range of the collagen is 500-935 grams, the content range of the hyaluronic acid is 1-5 grams, the content range of indigestible maltodextrin is 50-200 grams, the content range of Emblica officinalis is 2-30 grams, the content range of lingonberry extract is 1-20 grams, the content range of cherry blossom extract is 1-20 grams, the content range of oligomerized polyphenol is 0-20 grams, and the content range of flavoring ingredient is 0-175 grams.

2. The composition containing collagen according to claim 1, wherein the collagen is fish collagen or hydrolyzed fish collagen.

3. The composition containing collagen according to claim 1, wherein the hyaluronic acid is extracted from cockscomb.

4. The composition containing collagen for repairing and protecting skin wound according to claim 3, wherein the hyaluronic acid is extracted from cockscomb.

5. A use of the composition containing collagen as described in claim 1 for application to skin wound.
